(19) United States
(12) Patent Application Publication
(10) Pub. No.: US 2005/0192346 A1
(43) Pub. Date: Sep. 1, 2005

(54) Process for making nitric oxide releasing prodrugs of diaryl-2-(5H)-furanones as cyclooxygenase-2 inhibitors

(76) Inventors: Yao-Jun Shi, Edison, NJ (US); F. Conrad Engelhardt, Hoboken, NJ (US); Cameron John Cowden, Stanstead Abbots (GB); David A. Conlon, Plainsboro, NJ (US); Brenda Pipik, Edison, NJ (US)

Correspondence Address: MERCK AND CO., INC, P O BOX 2000, RAHWAY, NJ, 07065-0907, US

(21) Appl. No.: 11/066,676

(22) Filed: Feb. 25, 2005

Related U.S. Application Data
(60) Provisional application No. 60/549,126, filed on Mar. 1, 2004.

(51) Int. Cl. C07C 317/24; A61K 31/21

(52) U.S. Cl. 514/509; 558/482

(57) ABSTRACT

The invention encompasses a novel process for making compounds of Formula I 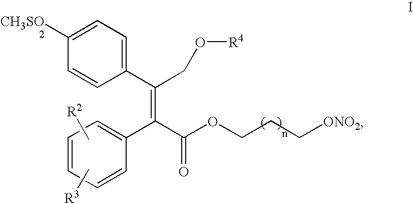 which are prodrugs of cyclooxygenase-2 selective inhibitors that convert in vivo to diaryl-2-(5H)-furanones and also liberate nitric oxide in vivo. As such, the compounds made by the present invention may be co-dosed with low-dose aspirin to treat chronic cyclooxygenase-2 mediated diseases or conditions, effectively reduce the risk of thrombotic cardiovascular events and potentially renal side effects and at the same time reduce the risk of GI ulceration or bleeding. The present invention describes an efficient and economical process for the preparation of 2,3-disubstituted (2Z)-4-acetoxybut-2-enoate derivatives that is useful for the production of kilogram quantities of material for preclinical and clinical use.

### BACKGROUND OF THE INVENTION

The present invention is directed to a process for making nitrosated prodrugs of cyclooxygenase-2 selective inhibitors that convert in vivo to diaryl-2-(5H)-furanones and also liberate nitric oxide in vivo. As such, the compounds made by the present invention may be co-dosed with low-dose aspirin to treat chronic cyclooxygenase-2 mediated diseases or conditions, effectively reduce the risk of thrombotic cardiovascular events and potentially renal side effects and at the same time reduce the risk of GI ulceration or bleeding.

The synthesis of a series of 2,3-disubstituted (2Z)-4-acetoxybut-2-enoic acids were reported in International Patent Publication WO 96/13483 (1996). Fallis et al., Tetrahedron Letters, vol. 41, no. 1, pp 17-20 (2000) described a method for preparing 2,3-disubstituted butenolides via a carbometallation route where the proposed intermediate (2Z)4-hydroxybut-2-enoic acids were not isolated. The nitration of an alcohol using a of acetic anhydride and nitric acid is well known, see: Black and Babers, Organic Syntheses, 19, pp 64-66 (1939); Malins, et al. J. Am. Chemists' Soc. vol. 41, no 1, pp 44-46 (1964). The spontaneous explosion of acetyl nitrate has been reported (Wibaut, Chemisch Weekblad, vol 39, pp 534 (1942). Kawashima et al., J. Med. Chem., vol. 36, pp 815-819 (1993) reported the alkylation of a carboxylic acid with ω-bromoalkyl nitrate. The reaction of an alkyl halide with silver nitrate is known to give a nitrate ester, see: Boschan et al., Chem. Rev., vol. 55, pp 485-510.

Although the synthetic methods disclosed in the above references suffice to prepare small quantities of material, they suffer from a variety of safety issues, low yields or lengthy processes that are not amenable to large scale synthesis. The present invention describes an efficient and economical process for the preparation of 2,3-disubstituted (2Z)-4-acetoxybut-2-enoate derivatives that is useful for the production of kilogram quantities of material for preclinical and clinical use.

### SUMMARY OF THE INVENTION

The invention encompasses a novel process for making compounds of Formula I 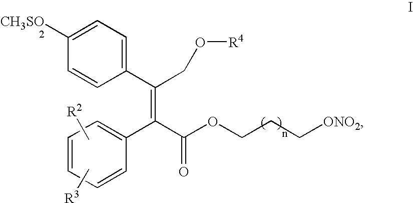
which are prodrugs of cyclooxygenase-2 selective inhibitors that convert in vivo to diaryl-2-(5H)-furanones and also liberate nitric oxide in vivo. As such, the compounds made by the present invention may be co-dosed with low-dose aspirin to treat chronic cyclooxygenase-2 mediated diseases or conditions, effectively reduce the risk of thrombotic cardiovascular events and potentially renal side effects and at the same time reduce the risk of GI ulceration or bleeding. The present invention describes an efficient and economical process for the preparation of 2,3-disubstituted (2Z)4-acetoxybut-2-enoate derivatives that is useful for the production of kilogram quantities of material for preclinical and clinical use.

### DETAILED DESCRIPTION OF THE INVENTION

The invention encompasses a process for making a compound of Formula I 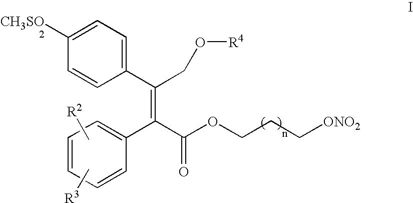
wherein:
n is an integer from 1 to 6;
R² and R³ each are independently selected from the group consisting of:
   (a) hydrogen and
   (b) halo; and
R⁴ is —C(O)—C₁₋₆alkyl;
comprising: reacting a compound of Formula A 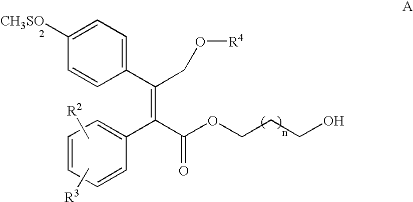
   with (a) an electrophilic nitrating reagent, or (b) activating the alcohol depicted in the Formula A to become a leaving group followed by displacement with a nitrate ion, said (a) or (b) conducted in a first organic solvent to yield a compound of Formula I,
   or alternatively reacting a compound of Formula A1 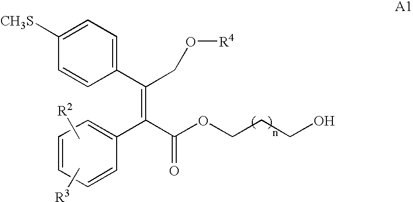
   with (a) an electrophilic nitrating reagent, or (b) activating of the alcohol depicted in the Formula A1 to become a leaving group followed by displacement with a nitrate ion, said (a) or (b) conducted in a first organic solvent to yield a compound of Formula Ia, 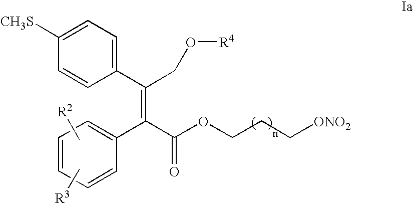
   and reacting the compound of Formula Ia with an oxidizing agent to yield a compound of Formula I.

The invention also encompasses the above process wherein: R² and R³ are both hydrogen; R⁴ is acetyl; the compound of Formula A or A1 is reacted with an electrophilic nitrating agent and the electrophilic nitrating agent is a combination of nitric acid and an anhydride of the formula [C₁₋₆alkyl(O)]₂O; and the first organic solvent is selected from the group consisting of: dichloromethane, dichloroethane, dichlorobenzene, nitromethane, acetonitrile and acetic acid. Within this embodiment the invention encompasses the above process wherein the anhydride is n-butyric anhydride and the first organic solvent is dichloromethane.

Another embodiment of the invention encompasses making the compound of Formula A by reacting a compound of Formula B 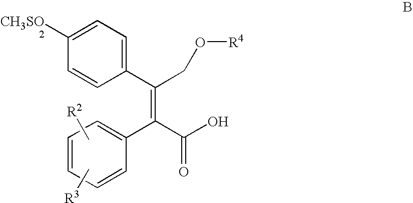
with a compound of Formula C 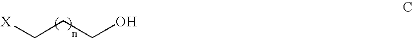
wherein X is a leaving group, in the presence of a base in a second organic solvent to yield a compound of Formula A,
or alternatively reacting a compound of Formula B1 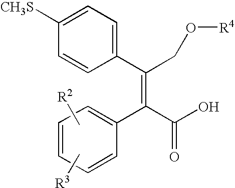
with a compound of Formula C 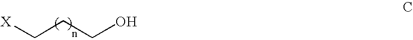
wherein X is a leaving group, in the presence of a base in a second organic solvent to yield a compound of Formula A1, and reacting the compound of Formula A1 with an oxidizing agent to yield a compound of Formula A.

Another embodiment of the invention encompasses making the compound of Formula A1 by reacting a compound of Formula B1 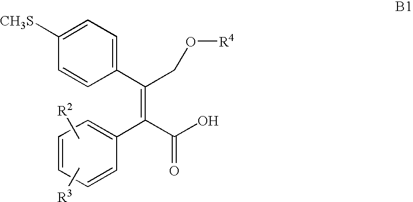
with a compound of Formula C 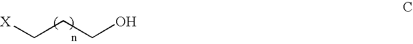
wherein X is a leaving group, in the presence of a base in a second organic solvent to yield a compound of Formula A1.

Another embodiment of the invention encompasses making compounds B and B1 according to the aforementioned process wherein: R² and R³ are both hydrogen; R⁴ is acetyl; X is selected from the group consisting of: bromo, chloro, iodo, tosyl, mesyl; the base is selected from the group consisting of: potassium carbonate, potassium bicarbonate, triethylamine, potassium tert-butoxide, potassium hexamethyldisilazide, cesium carbonate, sodium carbonate, and sodium bicarbonate; and the second organic solvent is selected from the group consisting of N,N-dimethylformamide, dimethyl sulfoxide, 1-methyl-2-pyrrolidinone, and N,N-dimethylacetamide. Within this embodiment, the invention encompasses this process wherein X is bromo; the base is potassium carbonate; and the second organic solvent is N,N-dimethylformamide.

Another embodiment of the invention encompasses a process for making a compound of Formula I 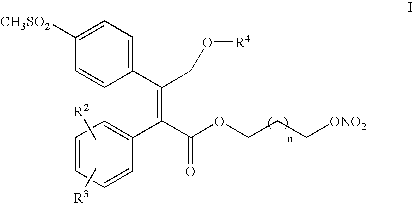
wherein:
n is an integer from 1 to 6;
R² and R³ each are independently selected from the group consisting of:
   (a) hydrogen and
   (b) halo; and
R⁴ is —C(O)—C₁₋₁₆alkyl;
   comprising reacting a compound of Formula B 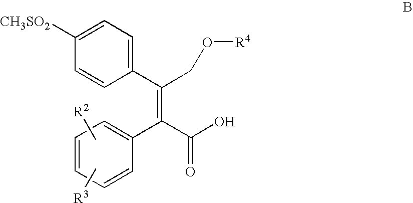
   with a compound according to Formula J 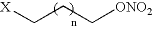
   wherein X is a leaving group, in the presence of a base in a second organic solvent to yield a compound of Formula I,
   or alternatively reacting a compound of Formula B1 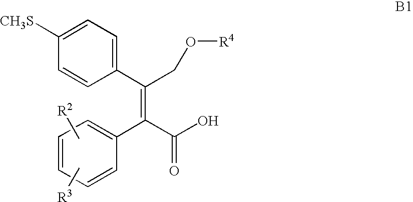
   with a compound according to Formula J 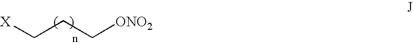
   wherein X is a leaving group, in the presence of a base in a second organic solvent to yield a compound of Formula Ia 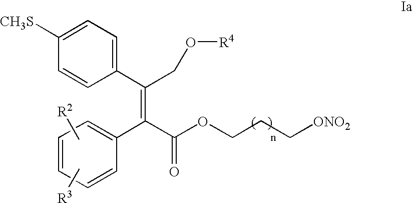
   and reacting the compound of Formula Ia with an oxidizing agent to yield a compound of Formula I. Within this embodiment, the invention encompasses this process wherein: R² and R³ are both hydrogen; R⁴ is acetyl; X is selected from the group consisting of: bromo, chloro, iodo, tosyl, mesyl; the base is selected from the group consisting of: potassium carbonate, potassium bicarbonate, triethylamine, potassium tert-butoxide, potassium hexamethyldisilazide, cesium carbonate, sodium carbonate, and sodium bicarbonate; and the second organic solvent is selected from the group consisting of N,N-dimethylformamide, dimethyl sulfoxide, 1-methyl-2-pyrrolidinone, and N,N-dimethylacetamide. Also within this embodiment, X is bromo; the base is potassium carbonate; and the second organic solvent is N,N-dimethylformamide.

The invention also encompasses a process for making the compound of Formula J 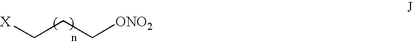
by reacting a compound of Formula C 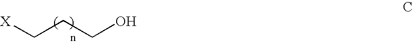
with (a) an electrophilic nitrating reagent or (b) activating the alcohol depicted in the Formula C to become a leaving group followed by displacement with a nitrate ion, said (a) or (b) conducted in a first organic solvent to yield a compound of Formula J. Within this embodiment, the compound of Formula C is reacted with an electrophilic nitrating agent and the electrophilic nitrating agent is a combination of nitric acid and an anhydride of the formula [C₁₋₆alkyl(O)]₂O; and the first organic solvent is selected from the group consisting of: dichloromethane, dichloroethane, dichlorobenzene, nitromethane, acetonitrile and acetic acid. Also within this embodiment, the anhydride of the formula [C₁₋₆alkyl(O)]₂O is acetic anhydride and the first organic solvent is dichloromethane.

The invention also encompasses a process for making the compound of Formula B by reacting a compound of Formula D 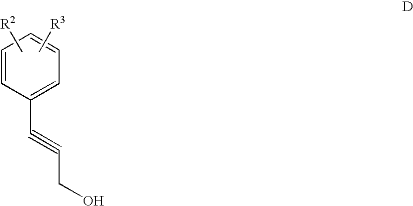
with a compound of Formula E 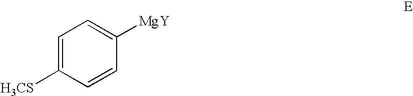
wherein Y is a halogen atom, and with carbon dioxide, an acetylating agent and a C₁-6 alkyl alkoxide in a third organic solvent to yield a compound of Formula B1, 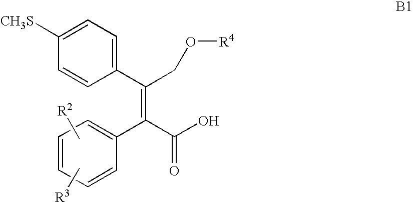
and isolating the compound of B1, or alternatively isolating the compound of Formula B1 as a salt, which can be subsequently converted to the free acid of Formula B1,
and reacting the compound of Formula B1 with an oxidizing agent to yield a compound of Formula B. Within this embodiment, the oxidizing agent is selected from the group consisting of: hydrogen peroxide, dimethyl dioxirane, potassium peroxymonosulfate, meta-chloroperbenzoic acid, sodium perborate and magnesium monoperoxyphthalate. Also within this embodiment, the oxidizing agent is hydrogen peroxide.

The invention also encompasses a process for making the compound of Formula B1 by reacting a compound of Formula D 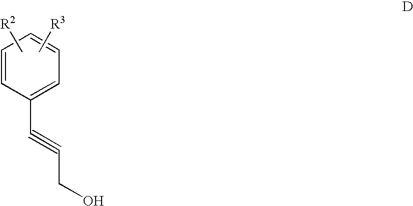
with a compound of Formula E 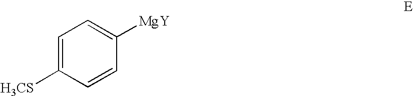
wherein Y is a halogen atom, and with carbon dioxide, an acetylating agent and a C₁₋₆ alkyl alkoxide in a third organic solvent to yield a compound of Formula B1, and isolating the compound of B1, or alternatively isolating the compound of Formula B1 as a salt, which can be subsequently converted to the free acid of Formula B1.

Another embodiment of the invention encompasses the above processes for making B and B1 wherein: R² and R³ are both hydrogen; R⁴ is acetyl; the acetylating reagent is selected from the group consisting of: acetic anhydride, acetyl chloride, acetyl bromide, and pyruvonitrile; the C₁₋₆ alkyl alkoxide is selected from the group consisting of: potassium t-butoxide, potassium ethoxide, sodium ethoxide and sodium methoxide; and the third organic solvent is selected from the group consisting of: tetrahydrofuran, cyclohexane, diethyl ether, toluene and dioxane. Also within this embodiment, Y is chloro; the acetylating agent is acetic anhydride; the C₁₋₆ alkyl alkoxide is potassium t-butoxide; and the third organic solvent is tetrahydrofuran.

Another embodiment of the invention encompasses the above processes wherein the compound of Formula B1 is isolated as a salt having the Formula F 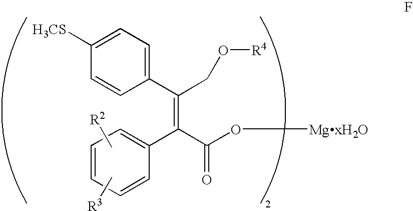
wherein x is an integer from 0 to 5
and subsequently converting the salt of Formula F into the acid of Formula B1.

Another embodiment of the invention encompasses the above processes, wherein prior to reacting with the compound of Formula E, the compound of Formula D is deprotonated with a compound of Formula H 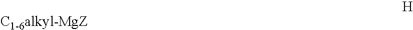
wherein Z is a halogen atom.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl, 1,1-dimethylethyl, cyclopropyl, cyclobutyl, cyclopentyl and cyclohexyl.

The term “electrophilic nitrating agent” means, for example, any acyl nitrate of the formula C₁₋₆alkyl C(O)ONO₂ which can be formed by the combination of nitric acid and an anhydride of the formula [C₁₋₆alkyl C(O)]₂O in a solvent such as dichloromethane.

The phrase “activating the alcohol to become a leaving group followed by displacement with a nitrate ion” can be accomplished, for example, by reaction of the alcohol under basic conditions with any of the following sulfonyl chlorides of the general formula RSO₂Cl or sulfonyl anhydrides of the formula [RSO_{2]}₂O where R=p-BrC₆H₄, C₆H₅, p-NO₂C₆H₄, p-CH₃C₆H₄, C₆H₅, CF₃, CH₃, CF₃C₆H₄ such as tosyl chloride, mesyl chloride, mesyl anhydride, nosyl chloride, brosyl chloride, or triflic anhydride. The resulting sulfonate leaving group can then be displaced by the addition a nitrate salt of the form M⁺ONO₂ such as silver nitrate or tetrabutylammonium nitrate.

The terms “anhydride” mean any organic carboxylic acid from which a water molecule has been removed, of the general formula [C₁₋₆alkyl C(O)]₂O.

The terms “first organic solvent,” “second organic solvent” and “third organic solvent” independently mean substantially non-reactive organic solvents or any mixture thereof. The term “first organic solvent” means, for example, dichloromethane, dichloroethane, dichlorobenzene, nitromethane, acetonitrile and acetic acid. The term “second organic solvent” means, for example, N,N-dimethylformamide, dimethyl sulfoxide, 1-methyl-2-pyrrolidinone, and N,N-dimethylacetamide. The term “third organic solvent” means, for example, tetrahydrofuran, cyclohexane, diethyl ether, toluene and dioxane.

The term “C₁₋₆alkyl alkoxide” means an organic alcohol of the form HOC₁₋₆alkyl in which the hydrogen of the hydroxyl group is replaced by a metal, for example, EtONa, t-BuOK.

The term “oxidizing agent” means a compound that readily yields oxygen, for example, hydrogen peroxide, dimethyldioxirane, potassium peroxymonosulfate (sold under the trade name OXONE®), meta-chloroperbenzoic acid or magnesium monoperoxyphthalate.

The term “leaving group” means any group that becomes displaced from carbon and, taking the electron pair with it, departs from the molecule. Examples of leaving groups include but are not limited to halogens (F, Cl, Br, and I) and alkyl or aryl sulfonates of the general formula [RSO₂O] where R=p-BrC₆H₄, C₆H₅, p-NO₂C₆H₄, p-CH₃C₆H₄, C₆H₅, CF₃, CH₃, CF₃C₆H₄ such as toslyate, mesylate, nosylate, brosylate, nonaflate, or triflate.

The term “acetylating agent” means, for example, an anhydride of the formula [C₁₋₆alkyl C(O)]₂O, an acyl halogen, such as acetyl chloride or acetyl bromide, or an acyl nitrile such as pyruvonitrile.

The term “halogen” or “halo” includes F, Cl, Br, and I.

The compounds of Formula I are prodrugs of cyclooxygenase-2 selective inhibitors which covert in vivo to diaryl-2-(5H)-furanones. The compounds also liberate nitric oxide in vivo. As such, the compounds of the present invention may be co-dosed with low-dose aspirin to treat chronic cyclooxygenase-2 mediated diseases or conditions, effectively reduce the risk of thrombotic cardiovascular events and potentially renal side effects and at the same time reduce the risk of GI ulceration or bleeding. Thus, patients with hypertension and cardiovascular disease, as well as potentially patients with renal insufficiency, would actively benefit from being administered compounds made by the present invention over NSAIDs and cyclooxygenase-2 selective inhibitors currently available. The activity of the compounds made by the process of this invention can be demonstrated in known assays that test for cyclooxygenase activity. For example, the human whole blood cyclooxgenase activity assay described in Brideau et al. (1996) Inflammation Res. 45: 68-74 may be employed. A model for probing gastric erosion is described in S. Fiorucci, et al., Gastroenterology, vol. 123, pp. 1598-1606, 2002 and M. Souza, et al., Am. J. Physiol. Gastrointest. Liver Physiol., vol. 285, pp. G54-G61, 2003.

The starting point for the present invention involves deprotonation of 3-aryl-2-propyn-1-ol with a Grignard reagent of the type C₁₋₆alkylMgZ (G). Use of this sacrificial Grignard means that a large excess of the functionalized aryl Grignard reagent (E) is not required. Subsequently, the aryl Grignard reagent (E) is added across the alkyne to give an intermediate vinyl Grignard that is trapped with carbon dioxide. An alkoxide is added at this point to remove excess carbon dioxide that is detrimental to the subsequent in situ acetylation. If an alkoxide is not added, then low yields of the desired product results. An acetylating agent is added and the product is then best isolated as its magnesium carboxylate salt (G). Fallis et al., Tetrahedron Letters, vol. 41, no. 1, pp 17-20 (2000) has previously demonstrated the synthesis of 2,3-disubstituted butenolide derivatives using related chemistry. In no case did Fallis teach the use a sacrifical Grignard nor report isolation of the acyclic 2,3-disubstituted (2Z)₄-alkoxybut-2-enoates. It is these compounds that are the basis of this patent application. These acyclic compounds rapidly undergo cyclization to afford butenolides and hence an important part of the current invention is the trapping of the acylic 2,3-disubstituted (2Z)₄-acetoxybut-2-enoic acids before cyclization can occur.

If the R¹ contains a sulfone group, the oxidation of the sulfide to the sulfone can be achieved at this stage or subsequently. If achieved now, then the oxidation is best performed on the free carboxylic acid and the magnesium salt is converted to the acid by treatment with a proton source such as AcOH.

Incorporation of the alkyl nitrate ester can be performed in one of two ways. The acid (B) can be alkylated with a haloalkanol (C) to afford an alcohol product of formula A which can then be nitrated using an electrophilic nitrating agent such as acetyl nitrate. More preferably, as acetyl nitrate is known to be explosive, this transformation is best achieved using butyroyl nitrate as an alternate nitrating agent prepared from butyric anhydride and nitric acid. The safety aspects of this combination have not been described previously. Alternatively, the alcohol could be converted to a leaving group which could be displaced with a nucleophilic nitrate source such as tetrabutylammonium nitrate.

Another route to the title compounds involves preparation of an ω-haloalkyl nitrate by nitration of the ω-haloalkanol. The ω-haloalkyl nitrate can then be reacted with the carboxylate and selective alkylation occurs at the halogen substituted site. This method is a more convergent approach.

The following exemplifies the present invention.

### PREPARATIVE EXAMPLE 1

### Synthesis of Common Intermediate

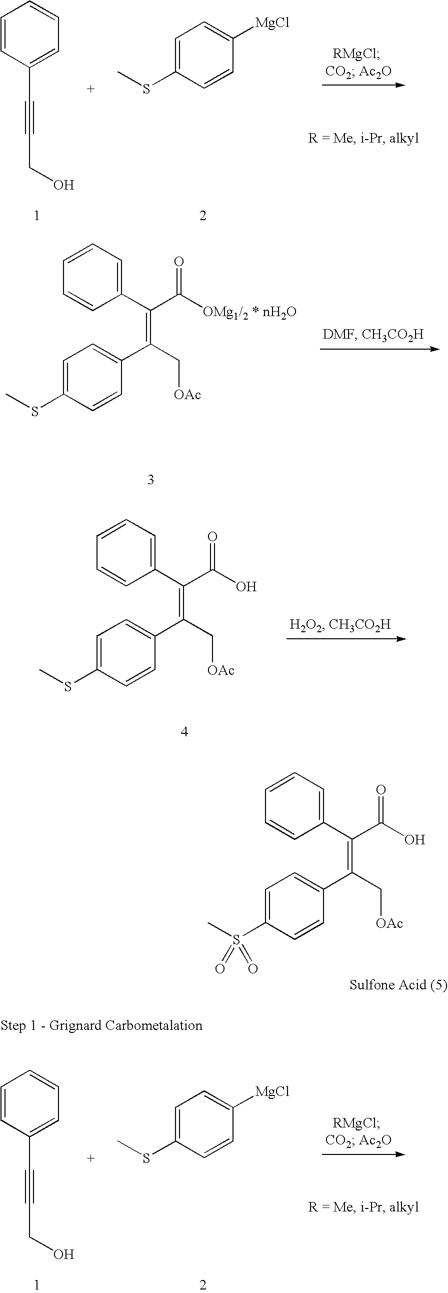

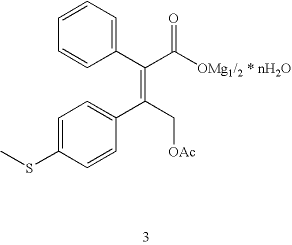

A flask is charged with 66.6 kg of THF and the vessel inerted with nitrogen. This was followed by the addition of 19.0 kg of 3-phenyl-2-propyn-1ol and then by a 16.6 kg THF flush. The batch was then cooled to approx. 5° C. and 49.8 kg of methyl magnesium chloride (3.0 M) was added slowly over 30 min. and achieved a final batch temperature between 25 and 30° C.

Then 92.2 kg of 4-thioanisole magnesium chloride was added (1.8 M) and the batch was heated to 65 to 70° C. under 2 to 10 psig back pressure. The batch was aged at this temperature for 3 h then cooled to 18° C. and vacuum pulled to 250 mmHg. Carbon dioxide (dry, 10.7 kg) was then charged slowly from a cylinder over 100 min to achieve a 5 psig pressure in the vessel. The batch was heated (30 to 35° C.) and aged further for 70 min.

The vessel pressure was vented and a series of pressure purges completed to remove residual carbon dioxide in the headspace. Then 64.7 kg of potassium tert-butoxide in THF (1.0 M sol) was charged followed by a 5.0 kg THF flush. The batch was aged at 32° C. for 30 min. A sample was then taken to confirm by IR that there was no residual carbon dioxide in solution.

The batch temperature was then adjusted to 23° C. and 28.6 kg of acetic anhydride was added, followed by a 10.0 kg THF flush. The batch was aged for 90 min before 303.9 kg of THF was added and the contents heated to 40° C. Then 16.1 kg of 45 wt % potassium hydroxide was added followed by a water flush (4.0 kg). The batch was aged at 40° C. for 7 h.

Next 277.8 kg of an aqueous 1.4 M magnesium chloride solution was added and the batch was aged for 15 min at 40° C. Agitation was then ceased and the two layers were allowed to settle. The layers were separated and then the organic layer was concentrated to 340 L at 20° C. under a vacuum of 125 mm Hg. Then 18.1 kg of water was added and finally, 542.8 kg of isopropyl acetate was added slowly over 4 h and 30 min at 20° C. to complete the crystallization of the batch. The batch was cooled to 0° C. then filtered and washed with 175 kg of water and 195 kg of cold (0° C.) isopropyl acetate. Filtration and drying provided 53.2 kg of the desired crystalline hydrated magnesium salt product (84% yield).

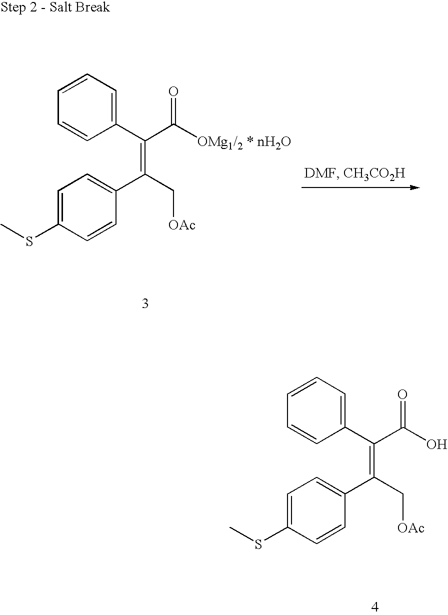

A solution of the magnesium salt 3 (2.63 kg corrected, 7.31 mol) in DMF (8 L) was slowly added to aqueous acetic acid (26 L, 2M, 56 mol) at 3540° C. The precipitated free acid 4 was isolated by filtration and the wet cake was washed with 20% aqueous DMF (6.6 L) and then twice with water (6.6 L). The product was dried at 40° C. under vacuum to yield 2.4 kg of the desired acid 4 as a tan crystalline solid (96% yield).

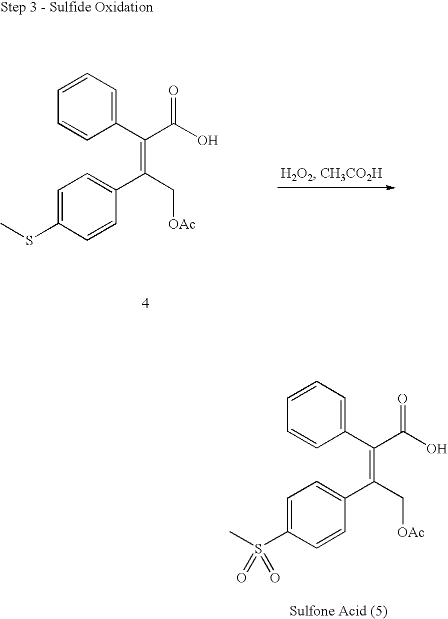

A mixture of acid 4 (2.56 kg, 7.30 mol) in acetic acid (24 L) was heated to 60° C. and hydrogen peroxide (3.26 L, 36.5 mol) was added over 15 min. After 2 h, the reaction was cooled to 40° C. and water (48 L) was added. The mixture was seeded and the temperature was held at 40° C. for 1 h then allowed to cool slowly to room temperature over 2 h. The batch was then cooled further to −10° C. and held at this temperature for 1 h. The product was isolated by filtration and washed with 7 L of water and dried under vacuum to afford sulfone acid 5 (2.46 kg, 89.8%) as a white crystalline solid.

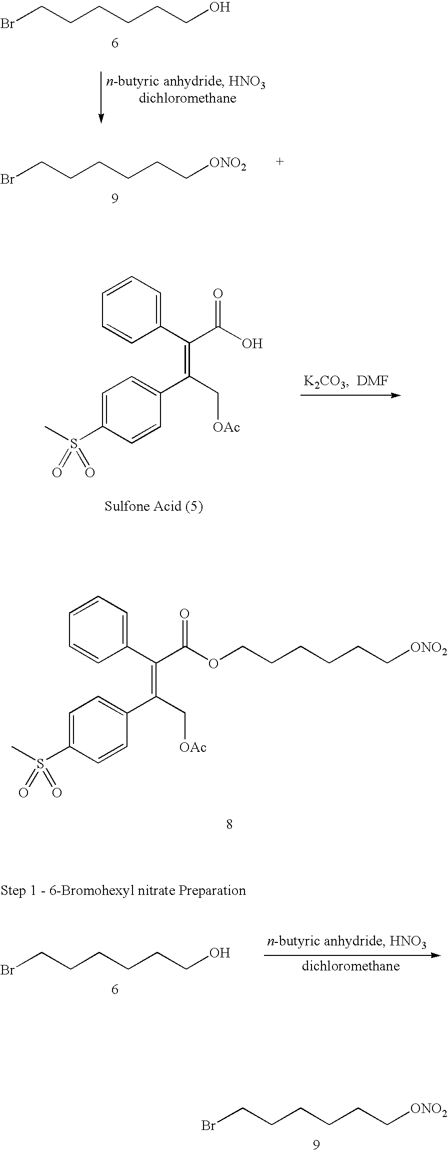

Nitric acid (90% w/w) (1.45 kg, 20.7 mol) was added over 1 h to a solution of acetic anhydride (2.53 kg, 24.8 mol) in dichloromethane (20 L) maintained at −10° C. This mixture was then aged at 0° C. for 1 h before a solution of 6-bromohexanol (2.50 kg, 13.8 mol) in dichloromethane (20 L) was added over 1.5 h maintaining the temperature below 0° C. The reaction was aged for 30 min and then quenched into K₂HPO₄ solution (10 L of 1 M). The organic layer was then treated with K₂HPO₄ solution (10 L of 1 M) and aged for 14 h before the layers were separated and the organic layer washed with urea solution (5 L of 10% w/w solution), water (20 L) and brine (10 L of saturated aqueous). The organic solution was then concentrated to afford 6-bromohexyl nitrate 9 (3.19 kg, 100 wt %, quant) as a colorless oil. 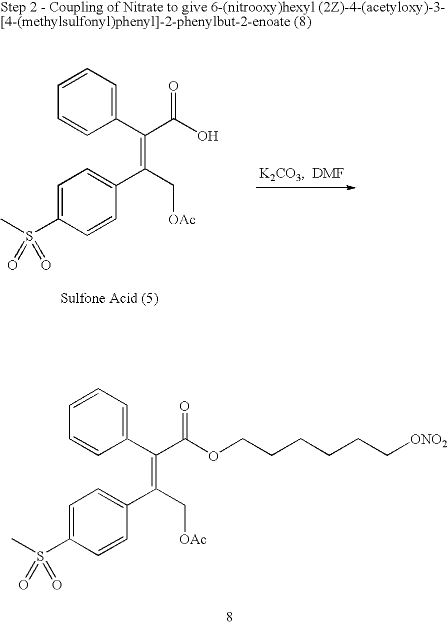
To a 100 L flask was charged 20 L of DMF, solid sulfone acid 5 (2.72 kg, 6.97 mol), bromohexyl nitrate 9 (3.99 kg, 17.2 mol), and 4.4 L of DMF to give a clear solution at room temperature. To this resulting solution was added powder K₂CO₃ (0.98 kg, 7.09 mol) in one portion at 20° C., followed by 2.0 L of DMF for rinse, and the mixture was then stirred at 20-22° C. for 2-3 h.

Next, ethyl acetate (30 L) was introduced and then cold water (30 L) added slowly to maintain the temperature <30° C. The mixture was stirred for 0.5 h and settled. The aqueous layer was separated and back-extracted with EtOAc (25 L). The combined organic layer was washed with water (2×20 L) and then saturated brine solution (26 L). The organic layer was concentrated in vacuo to ˜20 L, followed by addition of ˜20 L of n-heptane at 18-22° C. while it was aged for 1-2 h to provide a white slurry of the product. The remaining n-heptane was introduced over 1 h to afford a thick slurry. The slurry was cooled to 0-5° C. to reduce the supernatant concentration <1.5 mg/ml. It was then filtered and the cake was washed with cold pre-mixed EtOAc/n-heptane (⅓, 12 L) and air-dried at 23° C. under nitrogen for 12 h. The isolated white crystalline solid (3.56 kg) was obtained in 94% yield.

### EXAMPLE 2

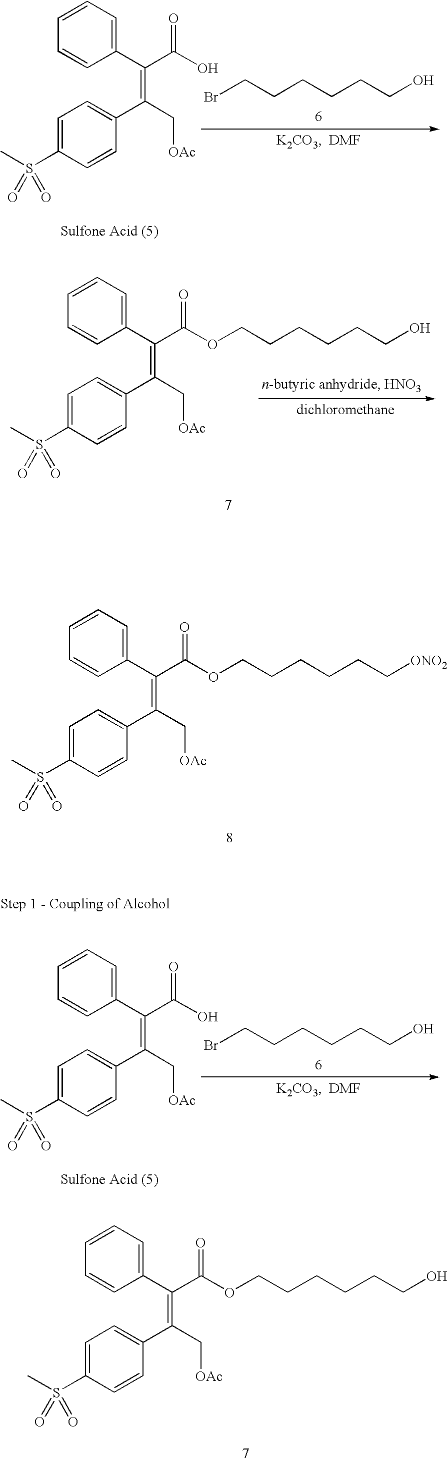

To a 50 L flask equipped with an overhead stirrer, thermocouple and nitrogen inlet was charged 9 L of DMF, bromohexanol 6, solid sulfone acid 5 and 2 L of DMF for rinse. To this was added powder K₂CO₃ in one portion at 20-22° C., followed by 2 L of DMF for rinse, and then stirred at 20-22° C. for 10 min and then heated to 40-45° C. for 3-5 h.

The reaction mixture was cooled to ˜20° C. and IPAc (26 L) was introduced and then ice cold water (19 L) added slowly to maintain the temperature <30° C. The mixture was stirred for 0.5 h before the aqueous layer was separated and back-extracted with IPAc (19 L). Combined organic layer was washed with water (2×19 L). The organic layer was concentrated in vacuo to ˜13 L, and flushed with 13 L of new IPAc. The resulting solution's concentration was adjusted to 170-180 mg/mL (˜15 L, KF<200 μg/ml). To this solution was added 5.5 L of n-heptane at 20-24° C. followed by addition of ˜25 g of the seed (˜1 wt % based on 95% yield), while it was aged for 1-2 h to provide a good seed-bed (supernatant ˜50 mg/ml) at 18-20° C. The remaining n-heptane (16.5 L) was introduced over 1-2 h and then aged for additional 8 h. The slurry was cooled to −5 to 0° C. then filtered and the cake was washed with cold pre-mixed IPAc/n-heptane (¼, 8 L) and air-dried at RT under nitrogen for 12 h. The isolated solid (2.58 kg, 95 wt %) was obtained in 90% yield.

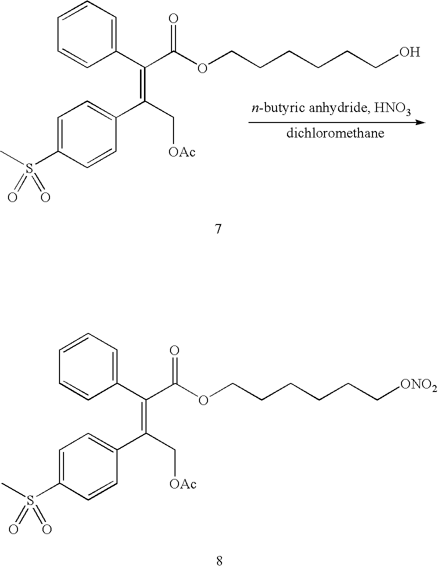

HNO₃ (344.6 mL, 7.33 mol) was added over 20 min to a cooled solution of n-butyric anhydride (1.38 kg, 8.69 mol) in dichloromethane (10 L) with the internal temperature remaining below 5° C. After aging for 2 h at 0° C., the solution was cooled to −15° C. and a solution of the alcohol 7 (2.20 kg, 4.64 mol) in dichloromethane (7.3 L) was added over 30 min maintaining the temperature below −10° C. The reaction was aged at −15, ° C. for 30 min. The reaction was quenched by addition of K₃PO₄ solution (8 L of 2 M aq. solution) then toluene (10 L) was added and the layers separated. The organic layer was washed with aqueous urea (20 L of 0.5%) then solvent switched to toluene (24 L final volume) followed by addition of heptane (2 L) at 35° C. to obtain a seed bed. Further addition of heptane (17 L) was made and filtration gave crude product. This material was recrystallized from toluene:heptane to give pure compound 8 as a white crystalline solid (2.05 kg, 90% yield).

1. A process for making a compound of Formula I 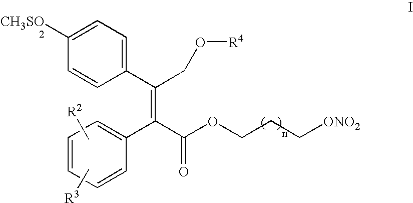
wherein:
n is an integer from 1 to 6;
R² and R³ each are independently selected from the group consisting of:
(a) hydrogen and
(b) halo; and
R⁴ is —C(O)—C₁₋₆alkyl;
comprising: reacting a compound of Formula A 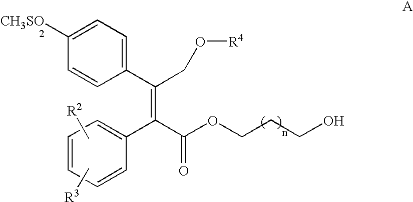
with (a) an electrophilic nitrating reagent, or (b) activating the alcohol depicted in the Formula A to become a leaving group followed by displacement with a nitrate ion, said (a) or (b) conducted in a first organic solvent to yield a compound of Formula I,
or alternatively reacting a compound of Formula A1 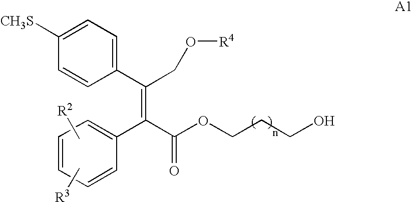
with (a) an electrophilic nitrating reagent, or (b) activating of the alcohol depicted in the Formula A1 to become a leaving group followed by displacement with a nitrate ion, said (a) or (b) conducted in a first organic solvent to yield a compound of Formula Ia, 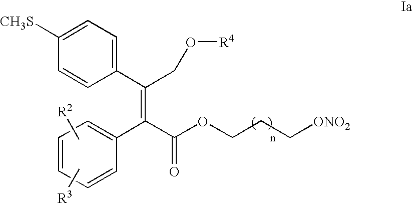
and reacting the compound of Formula Ia with an oxidizing agent to yield a compound of Formula I.

2. The process according to claim 1 wherein:
R² and R³ are both hydrogen;
R⁴ is acetyl;
the compound of Formula A or A1 is reacted with an electrophilic nitrating agent and the electrophilic nitrating agent is a combination of nitric acid and an anhydride of the formula [C₁₋₆alkyl(O)]₂; and
the first organic solvent is selected from the group consisting of: dichloromethane, dichloroethane, dichlorobenzene, nitromethane, acetonitrile and acetic acid.

3. (canceled)

4. The process according to claim 1 further comprising making the compound of Formula A by reacting a compound of Formula B 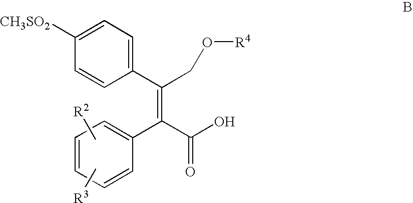
with a compound of Formula C 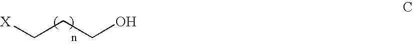
wherein X is a leaving group, in the presence of a base in a second organic solvent to yield a compound of Formula A,
or alternatively reacting a compound of Formula B1 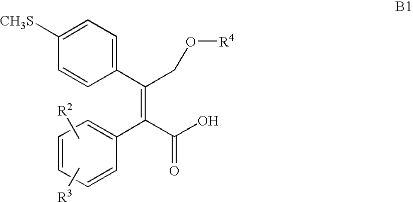
with a compound of Formula C 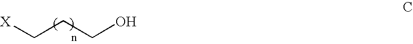
wherein X is a leaving group, in the presence of a base in a second organic solvent to yield a compound of Formula A1, and reacting the compound of Formula A1 with an oxidizing agent to yield a compound of Formula A.

5. The process according to claim 1 further comprising making the compound of Formula A1 by reacting a compound of Formula B1 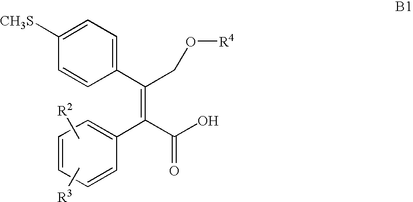
with a compound of Formula C 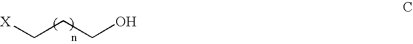
wherein X is a leaving group, in the presence of a base in a second organic solvent to yield a compound of Formula A1.

6. The process according to claim 4 wherein:
R² and R³ are both hydrogen;
R⁴ is acetyl;
X is selected from the group consisting of: bromo, chloro, iodo, tosyl, mesyl;
the base is selected from the group consisting of: potassium carbonate, potassium bicarbonate, triethylamine, potassium tert-butoxide, potassium hexamethyldisilazide, cesium carbonate, sodium carbonate, and sodium bicarbonate; and
the second organic solvent is selected from the group consisting of N,N-dimethylformamide, dimethyl sulfoxide, 1-methyl-2-pyrrolidinone, and N,N-dimethylacetamide.

7. (canceled)

8. The process according to claim 4 further comprising making the compound of Formula B by reacting a compound of Formula D 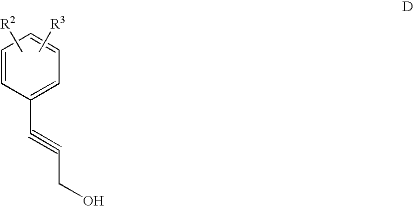
with a compound of Formula E 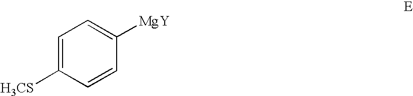
wherein Y is a halogen atom, and with carbon dioxide, an acetylating agent and a C₁₋₆ alkyl alkoxide in a third organic solvent to yield a compound of Formula B1, 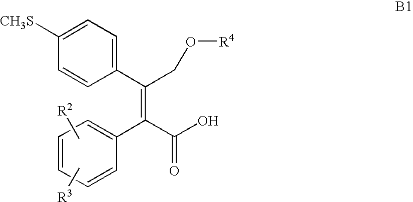
and isolating the compound of B1, or alternatively isolating the compound of Formula B1 as a salt, which can be subsequently converted to the free acid of Formula B1,
and reacting the compound of Formula B1 with an oxidizing agent to yield a compound of Formula B.

9. The process according to claim 8 wherein the oxidizing agent is selected from the group consisting of: hydrogen peroxide, dimethyl dioxirane, potassium peroxymonosulfate, meta-chloroperbenzoic acid, sodium perborate and magnesium monoperoxyphthalate.

10. (canceled)

11. The compound according to claim 4 further comprising making the compound of Formula B1 by reacting a compound of Formula D 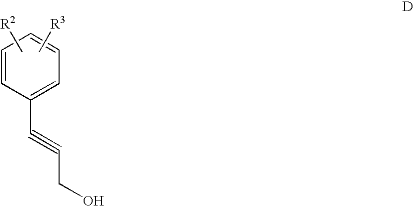
with a compound of Formula E 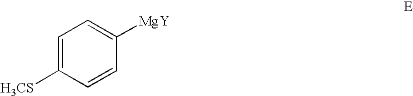
wherein Y is a halogen atom, and with carbon dioxide, an acetylating agent and a C₁₋₆ alkyl alkoxide in a third organic solvent to yield a compound of Formula B1, and isolating the compound of B1, or alternatively isolating the compound of Formula B1 as a salt, which can be subsequently converted to the free acid of Formula B1.

12. The process according to claim 8 wherein:
R² and R³ are both hydrogen;
R⁴ is acetyl;
the acetylating reagent is selected from the group consisting of: acetic anhydride, acetyl chloride, acetyl bromide, and pyruvonitrile;
the C₁₋₆ alkyl alkoxide is selected from the group consisting of: potassium t-butoxide, potassium ethoxide, sodium ethoxide and sodium methoxide; and
the third organic solvent is selected from the group consisting of: tetrahydrofuran, cyclohexane, diethyl ether, toluene and dioxane.

13. (canceled)

14. The process according to claim 8 wherein the compound of Formula B1 is isolated as a salt having the Formula F 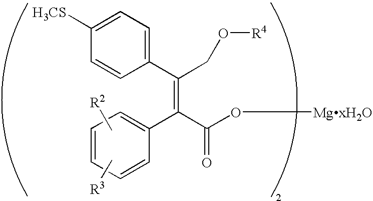
wherein x is an integer from 0 to 5
and subsequently converting the salt of Formula F into the acid of Formula B1.

15. The process according to claim 8, wherein prior to reacting with the compound of Formula E, the compound of Formula D is deprotonated with a compound of Formula H 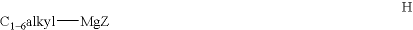
wherein Z is a halogen atom.

16. A process for making a compound of Formula I 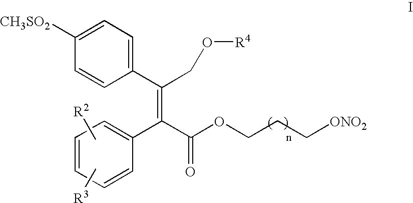
wherein:
n is an integer from 1 to 6;
R² and R³ each are independently selected from the group consisting of:
(a) hydrogen and
(b) halo; and
R⁴ is —C(O)—C₁₋₆alkyl;
comprising reacting a compound of Formula B 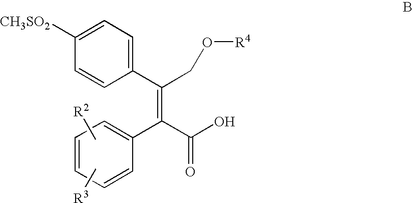
with a compound according to Formula J 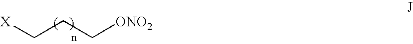
wherein X is a leaving group, in the presence of a base in a second organic solvent to yield a compound of Formula I,
or alternatively reacting a compound of Formula B1 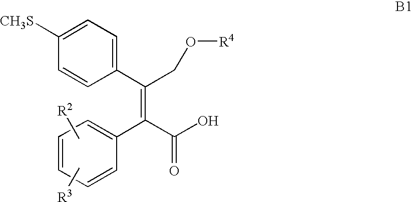
with a compound according to Formula J 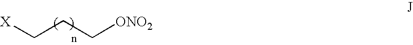
wherein X is a leaving group, in the presence of a base in a second organic solvent to yield a compound of Formula Ia 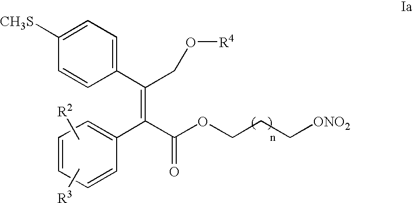
and reacting the compound of Formula Ia with an oxidizing agent to yield a compound of Formula I.

17. The process according to claim 16 wherein:
R² and R³ are both hydrogen;
R⁴ is acetyl;
X is selected from the group consisting of: bromo, chloro, iodo, tosyl, mesyl;
the base is selected from the group consisting of: potassium carbonate, potassium bicarbonate, triethylamine, potassium tert-butoxide, potassium hexamethyldisilazide, cesium carbonate, sodium carbonate, and sodium bicarbonate; and
the second organic solvent is selected from the group consisting of N,N-dimethylformamide, dimethyl sulfoxide, 1-methyl-2-pyrrolidinone, and N,N-dimethylacetamide.

18. (canceled)

19. A process according to claim 16 further comprising making the compound of Formula J 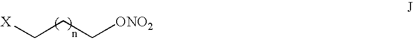
by reacting a compound of Formula C 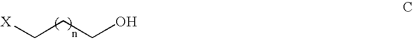
with (a) an electrophilic nitrating reagent or (b) activating the alcohol depicted in the Formula C to become a leaving group followed by displacement with a nitrate ion, said (a) or (b) conducted in a first organic solvent to yield a compound of Formula J.

20. The process according to claim 19 wherein:
R² and R³ are both hydrogen;
R⁴ is acetyl;
the compound of Formula C is reacted with an electrophilic nitrating agent and the electrophilic nitrating agent is a combination of nitric acid and an anhydride of the formula [C₁₋₆alkyl(O)]₂O; and
the first organic solvent is selected from the group consisting of: dichloromethane, dichloroethane, dichlorobenzene, nitromethane, acetonitrile and acetic acid.

21. (canceled)

22. The process according to claim 16 further comprising making the compound of Formula B by reacting a compound of Formula D 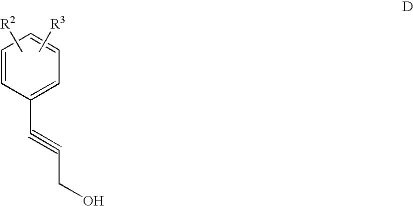
with a compound of Formula E 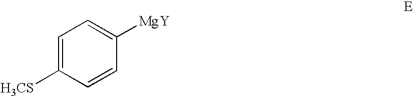
wherein Y is a halogen atom, and with carbon dioxide, an acetylating agent and a C₁₋₆ alkyl alkoxide in a third organic solvent to yield a compound of Formula B1, 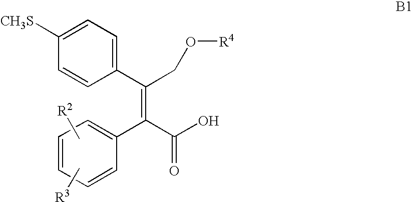
and isolating the compound of B1, or alternatively isolating the compound of Formula B1 as a salt, which can be subsequently converted to the free acid of Formula B1,
and reacting the compound of Formula B1 with an oxidizing agent to yield a compound of Formula B.

23. The process according to claim 22 wherein the oxidizing agent is selected from the group consisting of: hydrogen peroxide, dimethyl dioxirane, potassium peroxymonosulfate, meta-chloroperbenzoic acid, sodium perborate and magnesium monoperoxyphthalate

24. (canceled)

25. The process according to claim 16 further comprising making the compound of Formula B1 by reacting a compound of Formula D 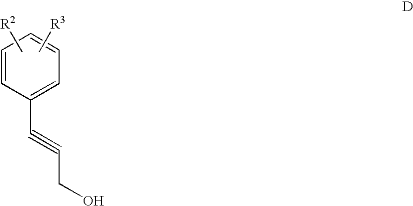
with a compound of Formula E 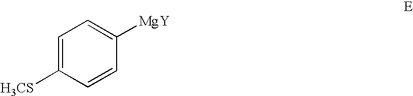
wherein Y is a halogen atom, and with carbon dioxide, an acetylating agent and a C₁₋₆ alkyl alkoxide in a third organic solvent to yield a compound of Formula B1, and isolating the compound of B1, or alternatively isolating the compound of Formula B1 as a salt, which can be subsequently converted to the free acid of Formula B1.

26. The process according to claim 22 wherein:
R² and R³ are both hydrogen;
R⁴ is acetyl;
the acetylating reagent is selected from the group consisting of: acetic anhydride, acetyl chloride, acetyl bromide, and pyruvonitrile;
the C₁₋₆ alkyl alkoxide is selected from the group consisting of: potassium t-butoxide, potassium ethoxide, sodium ethoxide and sodium methoxide; and
the third organic solvent is selected from the group consisting of: tetrahydrofuran, cyclohexane, diethyl ether, toluene and dioxane.

27. (canceled)

28. The process according to claim 22 wherein the compound of Formula B1 is isolated as a salt having the Formula F 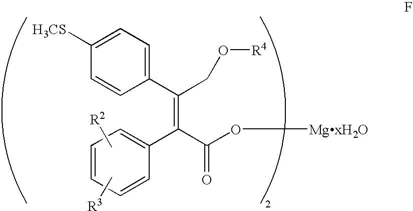
wherein x is an integer from 0 to 5
and subsequently converting the salt of Formula F into the acid of Formula B1.

29. The process according to claim 22, wherein prior to reacting with the compound of Formula E, the compound of Formula D is deprotonated with a compound of Formula H 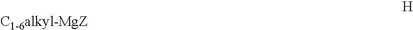
wherein Z is a halogen atom.
